(19) United States
(12) Patent Application Publication
(10) Pub. No.: US 2024/0172947 A1
(43) Pub. Date: May 30, 2024

(54) ELECTRONIC DEVICE AND METHOD OF ESTIMATING BLOOD FLOW INFORMATION USING THE SAME

(71) Applicants: SAMSUNG ELECTRONICS CO., LTD., Suwon-si (KR); KOREA ADVANCED INSTITUTE OF SCIENCE AND TECHNOLOGY, Daejeon (KR)

(72) Inventors: Bok Soon KWON, Suwon-si (KR); Kyeongha KWON, Daejeon (KR); Do Yun PARK, Seoul (KR); Young Min SIM, Daejeon (KR); Chulyoon JUNG, Daejeon (KR); Sang Kyu KIM, Suwon-si (KR); Sungho KIM, Suwon-si (KR); So Young LEE, Suwon-si (KR); Ho Taik LEE, Suwon-si (KR); Hong Soon RHEE, Suwon-si (KR)

(73) Assignees: SAMSUNG ELECTRONICS CO., LTD., Suwon-si (KR); KOREA ADVANCED INSTITUTE OF SCIENCE AND TECHNOLOGY, Daejeon (KR)

(21) Appl. No.: 18/127,445

(22) Filed: Mar. 28, 2023

(30) Foreign Application Priority Data
Nov. 25, 2022 (KR) 10-2022-0160847

(51) Int. Cl. A61B 5/026; A61B 5/00; A61B 5/0205; A61B 5/145

(52) U.S. Cl. CPC A61B 5/026; A61B 5/0008; A61B 5/0205; A61B 5/14532; A61B 5/6833; A61B 5/7246; A61B 5/7264; A61B 5/7275; A61B 5/7278; A61B 2562/0271; A61B 2562/043

(57) ABSTRACT

An electronic device for estimating body temperature may include: a sensor including a heat source, a pair of first temperature sensors horizontally spaced apart from each other at two opposing sides of the heat source, and a pair of second temperature sensors vertically spaced apart from the pair of first temperature sensors in a thickness direction of the electronic device; and at least one processor configured to estimate a blood temperature difference based on temperatures measured by the pair of first temperature sensors and the pair of second temperature sensors, and estimate blood flow information based on the estimated blood temperature difference.

### CROSS-REFERENCE TO RELATED APPLICATION(S)

This application claims priority from Korean Patent Application No. 10-2022-0160847, filed on Nov. 25, 2022, in the Korean Intellectual Property Office, the disclosure of which is incorporated herein by reference in its entirety.

### BACKGROUND

### 1. Field

Apparatuses and method according to one or more embodiments relate to obtaining blood flow information, such as blood flow velocity, by using the electronic device.

### 2. Description of the Related Art

Blood flow is an important physiological parameter that can provide valuable information about a person's cardiovascular health, such as stroke, hypertension, hyperlipidemia, and the like. Conventional methods for measuring blood flow, such as blood pressure cuffs or vascular ultrasonography, are invasive and/or bulky, making them unsuitable for daily use and for continuous monitoring. Accordingly, there is an increasing need for a wearable device capable of accurately and continuously measuring blood flow in daily life, which would provide valuable insights into a person's cardiovascular health and potentially lead to early detection and treatment of cardiovascular disease.

### SUMMARY

According to an aspect of the present disclosure, an electronic device may include: a sensor including a heat source, a pair of first temperature sensors horizontally spaced apart from each other at two opposing sides of the heat source, and a pair of second temperature sensors vertically spaced apart from the pair of first temperature sensors in a thickness direction of the electronic device: and at least one processor configured to: control the heat source to generate heat while the electronic device is in contact with a user: estimate a blood temperature difference between the two opposing sides of the heat source, based on temperatures measured by the pair of first temperature sensors and the pair of second temperature sensors; and estimate blood flow information of the user based on the blood temperature difference.

The at least one processor may be further configured to: calculate a first temperature difference between the temperatures measured by the pair of first temperature sensors and a second temperature difference between the temperatures measured by the pair of second temperature sensors: and estimate the blood temperature difference based on the first temperature difference and the second temperature difference.

The at least one processor may be further configured to: obtain an objective function that uses a relational expression between the first temperature difference and the blood temperature difference and a relational expression between the second temperature difference and the blood temperature difference: and obtain the blood temperature difference at which the objective function is minimized.

The sensor may further include a third temperature sensor horizontally spaced apart from the heat source, wherein the at least one processor may be further configured to correct the temperatures measured by the first temperature sensors based on temperature measured by the third temperature sensor.

The at least one processor is further configured to control an on-time period and an off-time period of the heat source according to a predetermined cycle, wherein the first temperature sensors and the second temperature sensors may be configured to measure temperatures during the off-time period of the heat source that follows the on-time period of the heat source.

The blood flow information may include at least one of a blood flow velocity, a blood flow direction, a core body temperature, and a blood glucose change.

The at least one processor may be further configured to estimate the blood flow velocity based on the blood temperature difference by using an estimation model that defines a correlation between the blood temperature difference and the blood flow velocity.

The at least one processor may be further configured to: estimate heat flux based on a difference between the temperatures measured by the first temperature sensor and the second temperature sensor which are disposed on at least one side of the heat source, and estimate the core body temperature based on the estimated heat flux.

Upon estimating the blood flow velocity, the at least one processor may be further configured to: calculate a difference between the estimated blood flow velocity and a reference blood flow velocity estimated at a reference time: and predict a change in blood glucose compared to the reference time based on the difference between the estimated blood flow velocity and a reference blood flow velocity.

At least one of the first temperature sensors and the second temperature sensors may be a thermistor.

The heat source has a diameter of 1 mm to 10 mm.

A distance between a center and the two opposing sides of the heat source is within 8.4 mm.

A distance between the pair of first temperature sensors and the pair of second temperature sensors is 0.4 mm to 10 mm.

The sensor may include a thermally insulating materials disposed between the first temperature sensors and between the second temperature sensors.

According to another aspect of the present disclosure, a method of estimating blood flow information by an electronic device, may include: controlling a heat source to generate heat while the electronic device is in contact with a user; measuring temperatures by using a pair of first temperature sensors horizontally spaced apart from each other at two opposing sides of the heat source, and a pair of second temperature sensors vertically spaced apart from the pair of first temperature sensors in a thickness direction of the electronic device; estimating a blood temperature difference between the two opposing sides of the heat source, based on the temperatures measured by the pair of first temperature sensors and the pair of second temperature sensors: and estimating blood flow information of the user based on the blood temperature difference.

The estimating of the blood temperature difference may include: calculating a first temperature difference between the temperatures measured by the pair of first temperature sensors and a second temperature difference between the temperatures measured by the pair of second temperature sensors: and estimating the blood temperature difference based on the first temperature difference and the second temperature difference.

The method may further include measuring a temperature by using a third temperature sensor horizontally spaced apart from the heat source, wherein the estimating of the blood temperature difference may include correcting the temperatures measured by the first temperature sensors based on the temperature measured by the third temperature sensor.

The controlling of the heat source may include: controlling an on-time period and an off-time period of the heat source according to a predetermined cycle, wherein the measuring of the temperatures may include measuring temperatures during the off-time period of the heat source that follows the on-time period of the heat source.

The estimating of the blood flow information may include estimating a blood flow velocity, included in the blood flow information, based on the estimated blood temperature difference by using an estimation model that defines a correlation between the blood temperature difference and the blood flow velocity.

According to another aspect of the present disclosure, a patch-type sensor may include: a contact pad configured to make contact with a skin surface: a heat source configured to apply heat to the skin surface when the contact pad makes contact with the skin surface; a pair of first temperature sensors horizontally spaced apart from each other at two opposing sides of the heat source: a pair of second temperature sensors vertically spaced apart from the pair of first temperature sensors in a depth direction of the patch-type sensor; and a communication interface configured to transmit temperature data, measured by the pair of first temperature sensors and the pair of the second temperature sensors, to an external electronic device, to enable the external electronic device to obtain blood flow information by estimating a blood temperature difference based on the temperature data.

### BRIEF DESCRIPTION OF THE DRAWINGS

The above and/or other aspects will be more apparent by describing certain example embodiments, with reference to the accompanying drawings, in which:

FIG. 1 is a block diagram illustrating an example of an electronic device for estimating blood flow information:

FIG. 2 is a diagram illustrating an example of a sensor structure;

FIG. 3 is a diagram explaining operation of a heat source of a sensor:

FIG. 4 is a block diagram illustrating another example of an electronic device for estimating blood flow information:

FIG. 5 is a view, as seen from above, of a sensor being in contact with an object:

FIG. 6 is a block diagram illustrating yet another example of an electronic device for estimating blood flow information;

FIGS. 7A and 7B are diagrams illustrating examples of outputting blood flow information:

FIG. 8 is a block diagram illustrating yet another example of an electronic device for estimating blood flow information:

FIG. 9 is a flowchart illustrating a method of estimating blood flow information according to an embodiment of the present disclosure:

FIG. 10 is a flowchart illustrating a method of estimating blood flow information according to another embodiment of the present disclosure:

FIGS. 11 to 14 are diagrams illustrating examples of structures of an electronic device according to embodiments of the present disclosure; and

FIGS. 15 and 16 are diagrams illustrating examples of obtaining blood flow information by using two or more devices operating in conjunction with each other.

### DETAILED DESCRIPTION

Example embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the example embodiments. However, it is apparent that the example embodiments can be practiced without those specifically defined matters. Also, well-known functions or constructions are not described in detail since they would obscure the description with unnecessary detail.

It will be understood that, although the terms “first,” “second,” etc. may be used herein to describe various elements, these elements should not be limited by these terms. These terms are only used to distinguish one element from another. Any references to singular may include plural unless expressly stated otherwise. In addition, unless explicitly described to the contrary, an expression such as “comprising” or “including” will be understood to imply the inclusion of stated elements but not the exclusion of any other elements. Also, the terms, such as ‘unit’ or ‘module’, etc., should be understood as a unit that performs at least one function or operation and that may be embodied as hardware, software, or a combination thereof.

Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, all of a, b, and c, or any variations of the aforementioned examples.

An electronic device according to various embodiments of the present disclosure which will be described below may be, for example, a wearable device, a smartphone, a tablet PC, a mobile phone, a video phone, an electronic book reader, a desktop computer, a laptop computer, a netbook computer, a workstation, a server, a PDA, a portable multimedia player (PMP), an MP3 player, a medical device, and a camera. The wearable device may include at least one of an accessory type wearable device (e.g., wristwatch, ring, bracelet, anklet, necklace, glasses, contact lens, or head mounted device (HMD)), a textile/clothing type wearable device (e.g., electronic clothing), a body-mounted type wearable device (e.g., skin pad or tattoo), and a body implantable type wearable device. However, the wearable device is not limited thereto and may include, for example, various portable medical measuring devices (antioxidant measuring device, blood glucose monitor, heart rate monitor, blood pressure measuring device, thermometer, etc.), magnetic resonance angiography (MRA), magnetic resonance imaging (MRI), computed tomography (CT), imaging system, ultrasonic system, etc.), and the like. However, the electronic device is not limited to the above devices

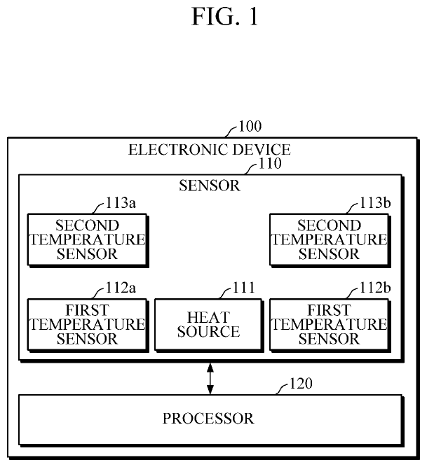

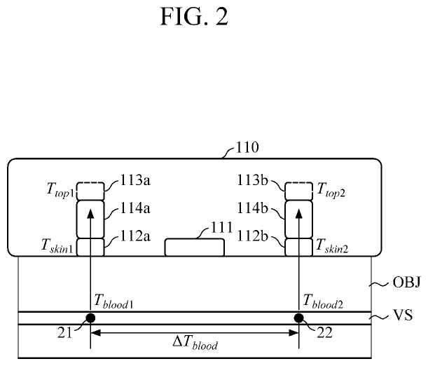

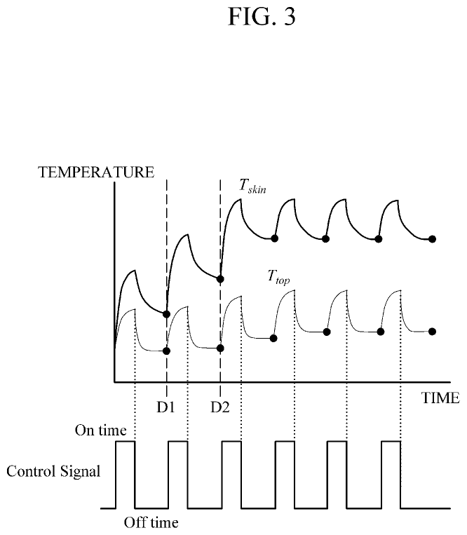

FIG. 1 is a block diagram illustrating an electronic device according to an embodiment of the present disclosure. FIG. 2 is a diagram illustrating an example of a sensor structure. FIG. 3 is a diagram explaining operation of a heat source of a sensor.

Referring to FIGS. 1 and 2, an electronic device 100 includes a sensor 110 and a processor 120.

The sensor 110 may include a heat source 111 and a plurality of temperature sensors 112a, 112b, 113a, and 113b. The heat source 111 and the plurality of temperature sensors 112a, 112b, 113a, and 113b may be mounted on a sensor board. In this case, the sensor board may be, for example, a flexible printed circuit board (FPCB) having flexible characteristics and capable of being bent in a predetermined shape. For example, the sensor board may have a size of 4 cm×2 cm, but is not limited thereto, and may be changed to various sizes according to the size of a form factor.

The heat source 111 may be disposed in the sensor 110 at a position adjacent to a contact position of an object OBJ, and may apply heat to the object OBJ during contact with the object OBJ. The heat source 111 may be made of a thin (and flat) metal film or wire, such as platinum, and may be formed by connecting one or more resistors. The heat source 111 may be connected to a power supply configured to provide power to the heat source 111 to generate heat, wherein the power may be current supplied from a General Purpose Input/Output (GPIO) of Bluetooth Low Energy SoC (BLE SoC), but examples of the power supply are not limited thereto.

The plurality of temperature sensors 112a, 112b, 113a, and 113b may be contact type temperature sensors (e.g., thermistors), but are not limited thereto, and some may be thermistors and the others may be non-contact type temperature sensors (e.g., infrared temperature sensors). A pair of first temperature sensors 112a and 112b may be horizontally spaced apart from each other at both sides of the heat source 111 so as to measure a surface temperature of the object OBJ. In addition, a pair of second temperature sensors 113a and 113b may be vertically spaced apart from the first temperature sensors 112a and 112b, respectively, so as to measure temperature at a position away from a surface of the object OBJ. However, the second temperature sensors 113a and 113b are not necessarily disposed in a line at positions vertically spaced apart from the first temperature sensors 112a and 112b in a thickness direction of the sensor 110, and the positions thereof are not limited thereto as long as the second temperature sensors 113a and 113b are disposed further away from the object OBJ than the first temperature sensors 112a and 112b.

The heat source 111 may have a diameter in a range of 1 mm to 10 mm. For example, in the case where the electronic device 100 is a smartwatch, the heat source 111 may have a diameter in a range of 1 mm to 5 mm, and the diameter may be, for example, 3 mm. In addition, in the case where the electronic device 100 is a patch-type device, the heat source 111 may have a diameter in a range of 1 mm to 10 mm, and the diameter may be, for example, 10 mm. The heat source 111 may have various shapes, such as a circular shape, a tetragonal shape, etc., with no particular limitation.

The first temperature sensors 112a and 112b, disposed at both sides of the heat source 111 in a horizontal direction, may be disposed within 8.4 mm from the center of the heat source 111. For example, in the case where the electronic device 100 is a smartwatch, each of the first temperature sensors 112a and 112b may be disposed at a distance of 4.2 mm+1 mm from the center of the heat source 111. In addition, in the case where the electronic device 100 is a patch-type device, each of the first temperature sensors 112a and 112b may be disposed at a distance of 8.2 mm from the center of the heat source 111. However, the distance is not limited thereto, and may vary according to the size of a form factor.

Further, the heat source 111 and the first temperature sensors 112a and 112b may be disposed at a distance of approximately 0.4 mm to 10 mm from the surface of the object OBJ during contact with the object OBJ. In addition, a distance between the first temperature sensors 112a and 112b and the second temperature sensors 113a and 113b in the thickness direction of the sensor 110 may be in a range of 0.4 mm to 10 mm. For example, in the case where the electronic device 100 is a smartwatch, the distance may range from 0.4 mm to 1.3 mm. However, the distance is not limited thereto, and may vary according to the size of a form factor.

As illustrated in FIG. 2, the sensor 110 may further include low thermal conductors 114a and 114b. The low thermal conductors 114a and 114b may be disposed between the first temperature sensors 112a and 112b and the second temperature sensors 113a and 113b and may maintain a constant distance between the first temperature sensors 112a and 112b and the second temperature sensors 113a and 113b. The low thermal conductor may be formed of, for example, an acrylic material, but is not limited thereto, and may be formed of, for example, insulation materials such as fiberglass, rock wool, and foam materials, plastics such as polystyrene and polyurethane, rubbers, elastomers, glass, air, certain ceramics, such as alumina and silica, and any other material with a thermal conductivity of 0.1 W/mK or less. The size or thermal conductivity of the low thermal conductor is not limited thereto. In addition, without using a separate thermally low conductive material, a structure filled with air having a relatively low thermal conductivity is also possible.

The processor 120 may be electrically connected to the sensor 110 via wire and/or wirelessly. The processor 120 may control the sensor 110 to obtain temperature data from the object OBJ. The object OBJ may be a surface of the wrist that is adjacent to the radial artery or an upper part of the wrist where venous blood or capillary blood passes, or a peripheral part of the body with high blood vessel density, such as fingers, toes, ears, or the like.

The processor 120 may receive temperature data from the sensor 110 and may estimate blood flow information by using the received temperature information. The blood flow information may include a variety of information related to vascular health, including a blood flow velocity, blood flow direction, core body temperature, blood glucose change, blood pressure change, arterial stiffness, vascular age, and the like.

In order to estimate the blood flow information, the processor 120 may control the heat source 111 to apply heat to the object OBJ. Once heat is applied to the object OBJ, a change in blood temperature is induced at two points 21 and 22 in a blood vessel VS of the object OBJ. In this case, the pair of first temperature sensors 112a and 112b at both sides of the heat source 111 may measure first temperatures Tₛᵢₙₖ₁ and Tₛᵢₙₖ₂ on the surface of the object OBJ, and the pair of second temperature sensors 113a and 113b may measure second temperatures Tₜₒₚ₁ and Tₜₒₚ₂ at positions relatively further away from the surface of the object OBJ than the first temperature sensors 112a and 112b.

In this case, when the second temperatures Tₜₒₚ₁ and Tₜₒₚ₂ are measured while the heat source 110 is still turned on, the heat source 110 may interfere with heat transfer from the first temperature sensors 112a and 112b toward the second temperature sensors 113a and 113b, thereby reducing measurement accuracy. Accordingly, the processor 120 may periodically turn on and off the heat source 111 as illustrated in FIG. 3 according to a control signal, and the first temperature sensors 112a and 112b and the second temperature sensors 113a and 113b may measure the first temperatures Tₛᵢₙₖ₁ and Tₛᵢₙₖ₂ and the second temperatures Tₜₒₚ₁ and Tₜₒₚ₂ in a stable state at a specific point D1 or D2 immediately before the heat source 110 is turned on, while the heat source 110 is turned off. In other words, the first temperatures Tₛᵢₙₖ₁ and Tₛᵢₙₖ₂ and the second temperatures Tₜₒₚ₁ and Tₜₒₚ₂ may be measured at the end of an off-time period, D1 or D2, which follows an on-time period.

The processor 120 may receive first temperature data and second temperature data measured by the first temperature sensors 112a and 112b and the second temperature sensors 113a and 113b, and may estimate blood flow information by using the received first temperature and second temperature. The processor 120 may estimate a blood temperature difference between the two points 21 and 22 of the blood vessel VS at both sides of the heat source 111, and may estimate a blood vessel velocity by using the blood temperature difference.

For example, the processor 120 may obtain a first temperature difference (ΔTₛₖᵢₙ=Tₛₖᵢₙ₂−Tₛₖᵢₙ₁) between the first temperatures Tₛᵢₙₖ₁ and Tₛᵢₙₖ₂ measured by the first temperature sensors 112a and 112b. In addition, the processor 120 may obtain a second temperature difference (ΔTₜₒₚ=Tₜₒₚ₂−Tₜₒₚ₁) between the second temperatures Tₜₒₚ₁ and Tₜₒₚ₂ measured by the second temperature sensors 113a and 113b. The temperatures Tₜₒₚ₁, Tₜₒₚ₂, Tₛᵢₙₖ₁, and Tₛᵢₙₖ₂ may be defined as shown in the following Equations 1 and 2, which may be rearranged to derive the following Equations 3 and 4 that define a relationship between the first temperature difference ΔTₛₖᵢₙ, the second temperature difference ΔTₜₒₚ, and blood temperature difference ΔT_{blood}=T_{blood2}−T_{blood1}.

$${T}_{skin 1} = {T}_{room} + \frac{{T}_{blood 1} - {T}_{r o o m}}{\frac{{t}_{t i s s u e}}{{k}_{t i s s u e}} + \frac{{t}_{d e v i c e}}{{k}_{d e v i c e}} + \frac{1}{h}} \times ( \frac{{t}_{d e v i c e}}{{k}_{d e v i c e}} + \frac{1}{h} ) \qquad [ Equation 1 ]$$

$${T}_{sink 2} may also be defined in a manner similar to {T}_{sink 1} , as follows .$$

$${T}_{s i n k 2} = {T}_{room} + \frac{{T}_{blood 2} - {T}_{r o o m}}{\frac{{t}_{t i s s u e}}{{k}_{t i s s u e}} + \frac{{t}_{d e v i c e}}{{k}_{d e v i c e}} + \frac{1}{h}} \times ( \frac{{t}_{d e v i c e}}{{k}_{d e v i c e}} + \frac{1}{h} )$$

$${T}_{top 1} = {T}_{room} + \frac{{T}_{b l o o d 1} - {T}_{r o o m}}{\frac{{t}_{t i s s u e}}{{k}_{t i s s u e}} + \frac{{t}_{d e v i c e}}{{k}_{d e v i c e}} + \frac{1}{h}} \times ( \frac{1}{h} ) \qquad [ Equation 2 ]$$

$${T}_{top 2} may also be defined in a manner similar to {T}_{top 1} , as follows .$$

$${T}_{top 2} = {T}_{room} + \frac{{T}_{blood 2} - {T}_{r o o m}}{\frac{{t}_{t i s s u e}}{{k}_{t i s s u e}} + \frac{{t}_{d e v i c e}}{{k}_{d e v i c e}} + \frac{1}{h}} \times ( \frac{1}{h} )$$

$$Δ {T}_{s k i n} = \frac{\frac{{t}_{d e v i c e}}{{k}_{d e v i c e}} + \frac{1}{h}}{\frac{{t}_{t i s s u e}}{{k}_{t i s s u e}} + \frac{{t}_{d e v i c e}}{{k}_{d e v i c e}} + \frac{1}{h}} \times ( Δ {T}_{b l o o d} ) \qquad [ Equation 3 ]$$

$$Δ {T}_{top} = \frac{\frac{1}{h}}{\frac{{t}_{t i s s u e}}{{k}_{t i s s u e}} + \frac{{t}_{d e v i c e}}{{k}_{d e v i c e}} + \frac{1}{h}} \times ( Δ {T}_{b l o o d} ) \qquad [ Equation 4 ]$$

In Equations 1 to 4, Tᵣₒₒₘ denotes ambient temperature around the sensor 110 and may denote, for example, temperature inside or outside the apparatus 100. Tᵣₒₒₘ is erased in the process of obtaining the first temperature difference ΔTₛₖᵢₙ and the second temperature difference ΔTₜₒₚ, such that there is no need for a separate temperature sensor for measuring ambient temperature. Subscripts tissue and device denote the object OBJ and a sensor layer 110, t_{device} and k_{device} denote the thickness and thermal conductivity of the sensor 110, respectively, and may be previously input values, and tₜᵢₛₛᵤₑ/kₜᵢₛₛᵤₑ denote tissue characteristics equivalent to the core body temperature of the object and tissue and kₜᵢₛₛᵤₑ may denote the thickness and thermal conductivity, respectively, h denotes a heat transfer coefficient, and ΔT_{blood} denotes the blood temperature difference. The first temperatures Tₛᵢₙₖ₁ and Tₛᵢₙₖ₂ and the second temperatures Tₜₒₚ₁ and Tₜₒₚ₂ denote values measured by the first temperature sensors 112a and 112b and the second temperature sensors 113a and 113b, the first temperature difference ΔTₛₖᵢₙ and the second temperature difference ΔTₜₒₚ are values obtained based on the first temperatures Tₛᵢₙₖ₁ and Tₛᵢₙₖ₂ and the second temperatures Tₜₒₚ₁ and Tₜₒₚ₂, in which tₜᵢₛₛᵤₑ/kₜᵢₛₛᵤₑ, h, and ΔT_{blood} are unknowns.

By using Equations 3 and 4 that define a relationship between the first temperature difference ΔTₛₖᵢₙ, the second temperature difference ΔTₜₒₚ, and the blood temperature difference ΔT_{blood}, the processor 120 may derive an objective function as shown in the following Equation 5.

$$\prod = {[ \frac{Δ {T}_{b l o o d}}{Δ {T}_{top}} \frac{\frac{1}{h}}{\frac{{t}_{t i s s u e}}{{k}_{t i s s u e}} + \frac{{t}_{d e v i c e}}{{k}_{d e v i c e}} + \frac{1}{h}} - 1 ]}^{2} + {[ \frac{Δ {T}_{b lood}}{Δ {T}_{s k i n}} \frac{\frac{{t}_{device}}{{k}_{device}} + \frac{1}{h}}{\frac{{t}_{t i s s u e}}{{k}_{t i s s u e}} + \frac{{t}_{device}}{{k}_{device}} + \frac{1}{h}} - 1 ]}^{2} \qquad [ Equation 5 ]$$

As described above, the processor 120 may obtain unknowns tₜᵢₛₛᵤₑ/kₜᵢₛₛᵤₑ, h, and ΔT_{blood} at which the derived objective function Π is minimized. The objective function Π may be stored in a storage of the electronic device 100, to obtain tₜᵢₛₛᵤₑ/kₜᵢₛₛᵤₑ, h, and ΔT_{blood} based on the objective function Π and the temperatures measured by the sensor 110. The objective function Π may be determined to be minimized when the output value of the objective function Π reaches a local minimum value or a predetermined minimum value.

Upon obtaining the blood temperature difference ΔT_{blood} as described above, the processor 120 may estimate a blood flow velocity by using the obtained blood temperature difference. For example, the processor 120 may obtain the blood flow velocity by inputting the blood temperature difference to an estimation model that defines a correlation between the blood temperature difference and the blood flow velocity. In this case, the estimation model may be a linear regression equation or a neural network-based model trained by deep learning.

In the above Equation 3, t, k, and h are all positive numbers, such that a relationship of ΔT_{blood}>ΔTₛₖᵢₙ is satisfied. In the following Equation 6, t, k, and h are irrelevant to a blood flow velocity f, such that a relationship of

$$\frac{d Δ {T}_{b l o o d}}{df} > \frac{d Δ {T}_{skin}}{df}$$

is satisfied, and it can be seen that the blood temperature difference ΔT_{blood} is more sensitive to a small flow velocity variation Δf than the first temperature difference ΔTₛₖᵢₙ.

$$\frac{d Δ {T}_{b lood}}{df} = \frac{\frac{{t}_{t i s s u e}}{{k}_{t i s s u e}} + \frac{{t}_{device}}{{k}_{device}} + \frac{1}{h}}{\frac{{t}_{device}}{{k}_{device}} + \frac{1}{h}} \times \frac{d Δ {T}_{s k i n}}{df} \qquad [ Equation 6 ]$$

In a conventional thermal mass flowmeter, only a temperature sensor on the skin surface is used, leading to a small temperature difference before and after the heat source, such that the accuracy of a blood flow velocity is reduced. As described above, in this embodiment, the blood temperature difference is estimated using the second temperature sensors 113a and 113b vertically disposed over the first temperature sensors 112a and 112b, and the blood flow velocity is estimated based on the blood temperature difference, thereby obtaining a high resolution compared to the case where only the temperature difference on the skin surface is used. In addition, as can be seen from the above Equation, there is no need for a separate sensor for measuring ambient temperature, thereby preventing an increase in device volume.

Further, the processor 120 may estimate a core body temperature by using the first temperature Tₛₖᵢₙ₁ and the second temperature Tₜₒₚ₁ which are measured by the first temperature sensor 112a and the second temperature sensor 113a disposed on one side of the heat source 111. For example, assuming that a flow of heat is current, a heat transfer characteristic of a material is resistance, and heat flux is voltage, the heat flow may be expressed in equation form according to Ohm's law (V=IR), and a temperature difference (Tₛₖᵢₙ₁−Tₜₒₚ₁) in the material may be estimated as the heat flux Q. Then, by combining the estimated heat flux Q with the surface temperature Tₛₖᵢₙ₁ of a body part to be measured, the processor 120 may estimate a core temperature at a body measurement position. Based on the relationship, Equation 7 for estimating the core body temperature at the body measurement position may be derived.

$${T}_{c o r e} = {T}_{s kin 1} + \frac{( {T}_{s kin 1} - {T}_{top 1} )}{ε} \qquad [ Equation 7 ]$$

Herein, ε denotes a predetermined coefficient, T_{core} denotes the core body temperature, Tₛₖᵢₙ₁ denotes the temperature measured by the first temperature sensor 112a, Tₜₒₚ₁ denotes the temperature measured by the second temperature sensor 113a, and Tₛₖᵢₙ₁−Tₜₒₚ₁ denotes the heat flux Q.

In another example, the processor 120 may estimate a core body temperature by inputting the first temperature, the heat flux, and the obtained blood flow velocity to a predefined core body temperature estimation model.

Likewise, the processor 120 may estimate a core body temperature by using the first temperature Tₛₖᵢₙ₂ and the second temperature Tₜₒₚ₂ measured by the first temperature sensor 112b and the second temperature sensor 113b which are disposed on the other side of the heat source 111, or the processor 120 may obtain, as the core body temperature, a statistical value (e.g., mean value) of core body temperature values measured by using all the temperature sensors at both sides of the heat source 111.

In addition, the processor 120 may estimate a blood flow direction. For example, the processor 120 may estimate the blood flow direction based on a positive or negative sign of the first temperature difference (ΔTₛₖᵢₙ=Tₛₖᵢₙ₂−Tₛₖᵢₙ₁). It can be estimated that if the first temperature difference ΔTₛₖᵢₙ is positive, the blood flows from the point 21 toward the point 22, and if the first temperature difference ΔTₛₖᵢₙ is negative, the blood flows from the point 22 toward the point 21.

Further, upon estimating the blood flow velocity, the processor 120 may obtain a variation in blood flow velocity by comparing the estimated blood flow velocity with a blood flow velocity estimated at a reference time, and may estimate information, such as arterial stiffness, blood glucose variation, vascular age, etc., based on the variation in blood flow velocity. For example, if the variation in blood flow velocity is less than or equal to a threshold value, the processor 120 may predict an increase in blood glucose levels, leading to an increase in blood viscosity. Further, if the variation in blood flow velocity is less than or equal to the threshold value, the processor 120 may predict an increase in arterial stiffness. As described above, upon predicting the increase in blood glucose levels and/or arterial stiffness based on the variation in blood flow velocity, the processor 120 may generate and provide warning information for a user.

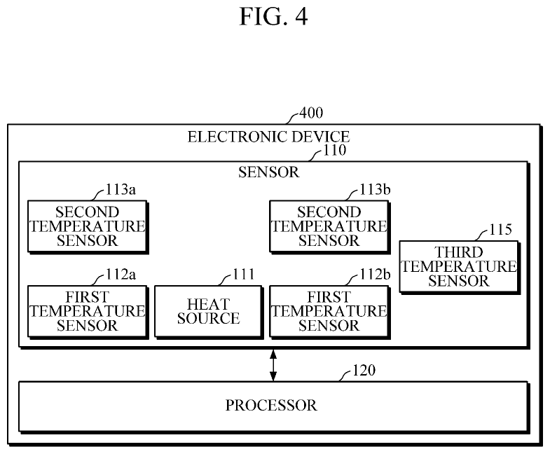

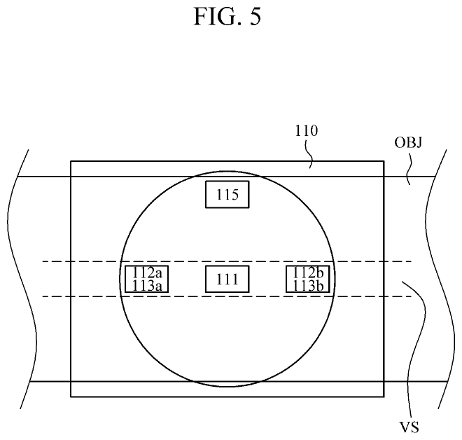

FIG. 4 is a block diagram illustrating another example of an electronic device for estimating blood flow information. FIG. 5 is a view, as seen from above, of a sensor being in contact with an object.

Referring to FIG. 4, an electronic device 400 includes the sensor 110 and the processor 120. The sensor 110 may include the heat source 111, the first temperature sensors 112a and 112b, the second temperature sensors 113a and 113b, and the third temperature sensor 115. Further, the electronic device 400 may further include low thermal conductors disposed between the first temperature sensors 112a and 112b and the second temperature sensors 113a and 113b as described above.

The heat source 111 and the pair of first temperature sensors 112a and 112b may be arranged side by side at a contact position with the object. The pair of second temperature sensors 113a and 113b may be vertically disposed over the pair of first temperature sensors 112a and 112b at a position further away from the object.

As illustrated in FIG. 5, when the sensor 110 or the electronic device 100 including the sensor 110 makes contact with the object OBJ, the heat source 111, the pair of first temperature sensors 112a and 112b, and the pair of second temperature sensors 113a and 113b may be in contact at a position where the blood vessel VS passes, and the third temperature sensor 115 may be in contact at a position where the blood vessel VS does not pass.

As described above, when a blood temperature difference is induced by operating the heat source 111, the first temperature sensors 112a and 112b and the second temperature sensors 113a and 113b may measure first temperatures on the surface at two points where the blood vessel VS of the object OBJ passes and measure second temperatures of a space away from the surface. The third temperature sensor 115 may measure a third temperature on the surface of the object where the blood vessel does not pass. The temperature sensors 112a, 112b, 113a, and 113b may be thermistors.

The processor 120 may correct the first temperatures on the skin surface, which are measured by the first temperature sensors 112a and 112b, by using the third temperature measured by the third temperature sensor 115. For example, the processor 120 may obtain the corrected first temperatures by subtracting the third temperature from each of the first temperatures, but is not limited thereto. The processor 120 may estimate a blood temperature difference based on the corrected first temperatures and the second temperatures as described above, and may estimate the blood flow velocity by using the estimated blood temperature difference. In addition, the processor 120 may monitor a variety of cardiovascular information, such as the core body temperature, blood glucose change, arterial stiffness, vascular age, etc., and may provide a user with warning information and the like.

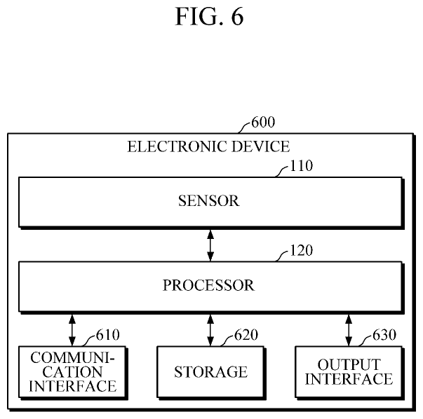

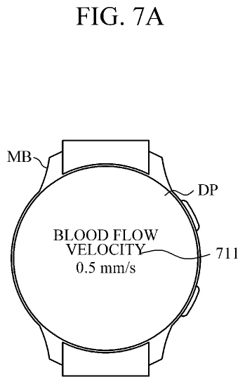

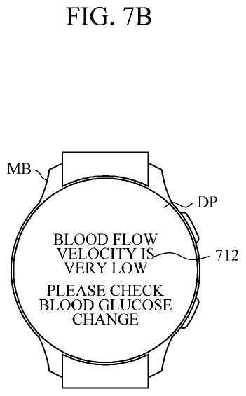

FIG. 6 is a block diagram illustrating yet another example of an electronic device for estimating blood flow information. FIGS. 7A and 7B are diagrams illustrating examples of outputting blood flow information.

Referring to FIG. 6, an electronic device 600 may further include a communication interface 610, a storage 620, and an output interface 630, in addition to the sensor 110 and the processor 120.

The sensor 110 includes the heat source 111, the first temperature sensors 112a and 112b, the second temperature sensors 113a and 113b, and/or the third temperature sensor 115, as illustrated in FIG. 1 or FIG. 4.

The processor 120 may control the heat source 111, may estimate a blood temperature difference by using the temperatures measured by the plurality of temperature sensors, and may estimate blood flow information, such as a blood flow velocity and the like, by using the estimated blood temperature difference.

The communication interface 610 may transmit temperature data, measured by the plurality of temperature sensors, to another electronic device. Further, the communication interface 610 may transmit the blood flow information, such as the blood temperature difference, blood flow velocity, etc., which are estimated by the processor 120, to another electronic device. In addition, the communication interface 610 may receive temperature data measured by another electronic device or sensor.

The communication interface 610 may communicate with another external device by using various wired or wireless communication techniques, such as Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra-Wideband (UWB) communication, Ant+ communication, WIFI communication, Radio Frequency Identification (RFID) communication, 3G, 4G, and 5G communications, and the like. However, this is merely exemplary and is not intended to be limiting.

The storage 620 may store various instructions. In addition, the storage 620 may store various data necessary for estimating blood flow information, including a blood flow velocity estimation model, a heat transfer coefficient, a core body temperature estimation model, criteria for controlling a heat source, and the like. Further, the storage 620 may store user characteristic information, such as a user's age, height, weight, exercise information, health information, and the like. Moreover, the storage 620 may store various data processed and generated by the electronic device 600.

The storage 620 may include at least one storage medium of a flash memory type memory, a hard disk type memory, a multimedia card micro type memory, a card type memory (e.g., an SD memory, an XD memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like, but is not limited thereto.

The output interface 630 may output data processed by the electronic device 600, for example, information including the first temperature, second temperature, blood temperature difference, blood flow velocity, blood flow direction, core body temperature, blood glucose change, arterial stiffness, etc., or warning information in a visual/non-visual manner by using a display, an audio output module, a haptic module, and the like. For example, as illustrated in FIG. 7A, the output interface 630 may output the blood flow velocity information estimated by the processor 120. In this case, depending on whether the blood flow velocity is normal, the output interface 630 may output the blood flow velocity information in different colors, and may further output warning information as illustrated in FIG. 7B.

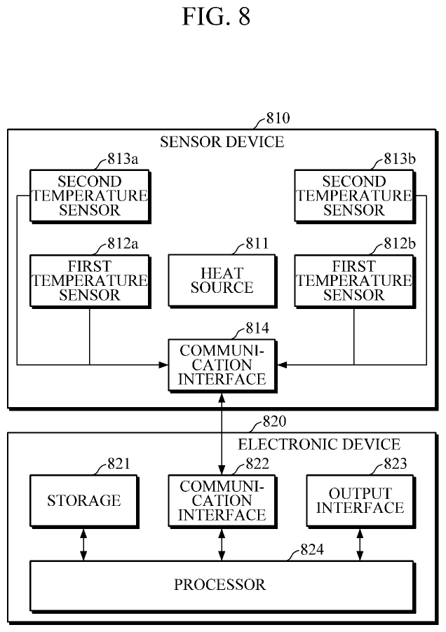

FIG. 8 is a block diagram illustrating yet another example of an electronic device for estimating blood flow information. Referring to FIG. 8, the electronic device for estimating blood flow information is formed as two or more separate devices 810 and 820 which may interwork with each other to estimate blood flow information.

The sensor device 810 is a device for measuring temperature data from an object and may include a heat source 811, first temperature sensors 812a and 812b, second temperature sensors 813a and 813b, and a communication interface 814. In addition, the sensor device 810 may further include a third temperature sensor. The sensor device 810 may be, for example, a patch-type sensor which comes into contact with an object. However, the sensor device 810 is not limited thereto, and may include various electronic device, such as a smartwatch, an ear-wearable device, a smartphone, and the like.

The communication interface 814 may include a BLE communication protocol, but is not limited thereto. The communication interface 814 may be connected to the heat source 811 and may apply, for example, a current from a GPIO of BLE SoC, to the heat source 811. The communication interface 814 may receive temperature data from the temperature sensors 812a, 812b, 813a, and 813b, and may transmit the receive temperature data to the electronic device 820. In this case, the communication interface 814 may convert the received temperature data into digital information through a Variable Gain Amplifier (VGA) and an Analog-to-Digital Converter (ADC), and may transmit the digital information to the electronic device 820.

The electronic device 820 may include a storage 821, a communication interface 822, an output interface 823, and a processor 824. The electronic device 820 may be a smartphone, a smartwatch, a table PC, a desktop computer, a medical institution server, etc., and is not particularly limited.

The communication interface 822 may receive temperature data from the sensor device 810 by using various communication techniques described above, and may transmit the temperature data to the processor 824. In addition, the communication interface 822 may transmit, to the sensor device 810, a signal for controlling the sensor device 810 under the control of the processor 824.

The processor 824 may estimate a blood temperature difference and blood flow information based on the received temperature data, as described above. The electronic device 820 may further include the sensor 110 of the electronic device 100 of FIG. 1 or FIG. 4. In this case, the processor 824 may control the sensor device 810 to obtain temperature data at a first position (e.g., in the vicinity of the upper arm) of an object and may obtain temperature data at a second position (e.g., wrist) of the object by using a sensor mounted therein, and may obtain respective blood flow values by using each of the temperature data measured at the first position and the temperature data measured at the second position, as described above. Further, the processor 824 may compare the obtained respective blood flow values or may calculate a statistical value (e.g., mean value) of the respective blood flow values.

The storage 821 may store temperature data, blood temperature difference, and/or blood flow information, etc., and the output interface 823 may output information generated by the processor 824, for example, the blood temperature difference, blood flow information, warning information, and the like.

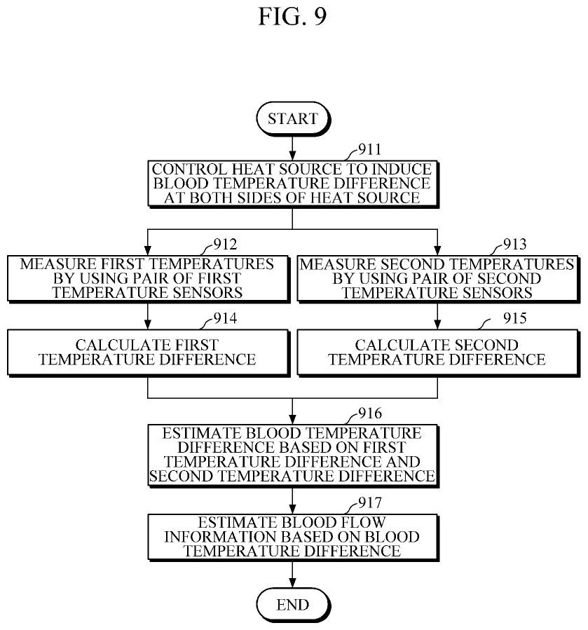

FIG. 9 is a flowchart illustrating a method of estimating blood flow information according to an embodiment of the present disclosure. The method of FIG. 9 may be performed by the electronic device of FIG. 1 or FIG. 6, which is described in detail above, and thus will be briefly described below in order to avoid redundancy.

First, the electronic device may control the heat source of the sensor in operation 911. The heat source may be disposed in the sensor at a position adjacent to a contact position of an object. The electronic device may periodically control the heat source, and by operating the heat source, the electronic device may induce a blood temperature difference between two points of the blood vessel at both sides of the heat source.

Then, the electronic device may measure first temperatures on the surface of the object by using the pair of first temperature sensors disposed side by side in a horizontal direction of the heat source in the sensor in operation 912. In addition, the electronic device may measure second temperatures at a position further away from the surface of the object by using the pair of second temperature sensors vertically spaced apart from the pair of first temperature sensors in the sensor in operation 913.

Subsequently, the electronic device may calculate, in operation 914, a first temperature difference between the first temperatures measured by the pair of first temperature sensors in operation 912. In addition, the electronic device may calculate, in operation 915, a second temperature difference between the second temperatures measured by the pair of second temperature sensors in operation 913.

Next, the electronic device may estimate a blood temperature difference based on the first temperature difference and the second temperature difference in operation 916. There is a relationship between the first and second temperature differences and the blood temperature difference as shown in Equations 3 and 4, and the electronic device may derive the objective function shown in Equation 5 based on the relationship, and may obtain a blood temperature difference at which the objective function is minimized.

Then, the electronic device may estimate blood flow information based on the blood temperature difference in operation 917. For example, the electronic device may estimate a blood flow velocity based on the blood temperature difference by using a blood flow velocity estimation model which defines a correlation between the blood temperature difference and the blood flow velocity. Further, as described above, the electronic device may obtain information, such as blood flow direction, core body temperature, and the like. Based on a relationship between a change in blood flow velocity and a change in blood glucose and arterial stiffness, the electronic device may monitor the change in blood glucose and arterial stiffness according to the change in blood flow velocity, and may generate warning information.

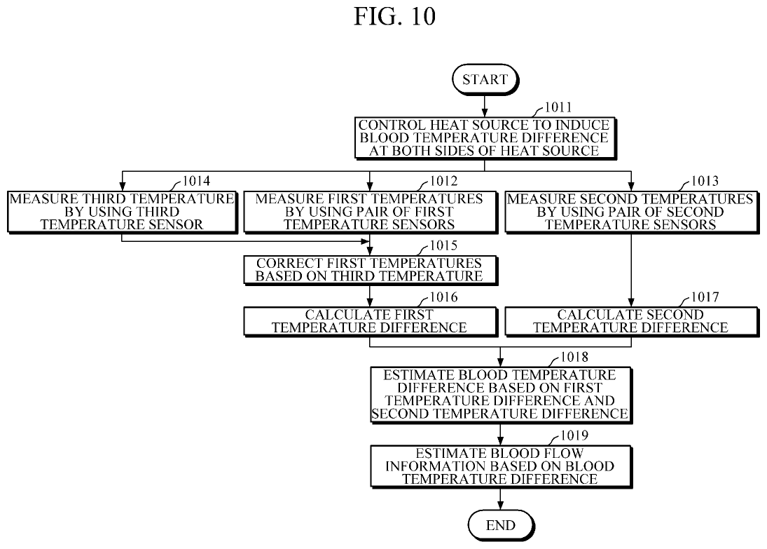

FIG. 10 is a flowchart illustrating a method of estimating blood flow information according to another embodiment of the present disclosure. The method of FIG. 10 may be performed by the electronic device of FIG. 4 or FIG. 6, which is described in detail above, and thus will be briefly described below in order to avoid redundancy.

First, the electronic device may control the heat source of the sensor to induce a blood temperature difference at both sides of the heat source in operation 1011. The electronic device may periodically control the heat source.

Then, the electronic device may measure first temperatures on the surface of the object by using the pair of first temperature sensors disposed side by side in a horizontal direction of the heat source in the sensor in operation 1012. In addition, the electronic device may measure second temperatures at a position further away from the surface of the object by using the pair of second temperature sensors vertically spaced apart from the pair of first temperature sensors in the sensor in operation 1013. Further, by using a third temperature sensor, the electronic device may measure a third temperature on the surface of the object at a position where the blood vessel of the object does not pass in operation 1014.

Subsequently, the electronic device may correct the first temperatures based on the third temperature in operation 1015. For example, the electronic device may obtain the corrected first temperatures by subtracting the third temperature from each of the first temperatures.

Next, the electronic device may calculate, in operation 1016, a first temperature difference between the first temperatures corrected in operation 1015. In addition, the electronic device may calculate, in operation 1017, a second temperature difference between the second temperatures measured by the pair of second temperature sensors in operation 1013.

Then, the electronic device may estimate a blood temperature difference based on the first temperature difference and the second temperature difference in operation 1018. There is a relationship between the first and second temperature differences and the blood temperature difference as shown in Equations 3 and 4, and the electronic device may derive the objective function shown in Equation 5 based on the relationship, and may obtain a blood temperature difference at which the objective function is minimized.

Subsequently, the electronic device may estimate blood flow information based on the blood temperature difference in 1019. For example, the electronic device may estimate a blood flow velocity based on the blood temperature difference by using a blood flow velocity estimation model which defines a correlation between the blood temperature difference and the blood flow velocity. Further, as described above, the electronic device may obtain information, such as blood flow direction, core body temperature, and the like. Based on a relationship between a change in blood flow velocity and a change in blood glucose and arterial stiffness, the electronic device may monitor the change in blood glucose and arterial stiffness according to the change in blood flow velocity, and may generate warning information.

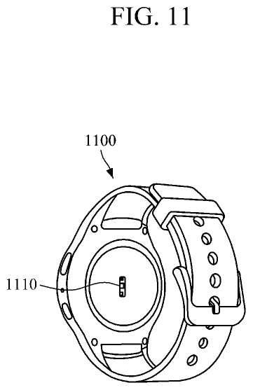

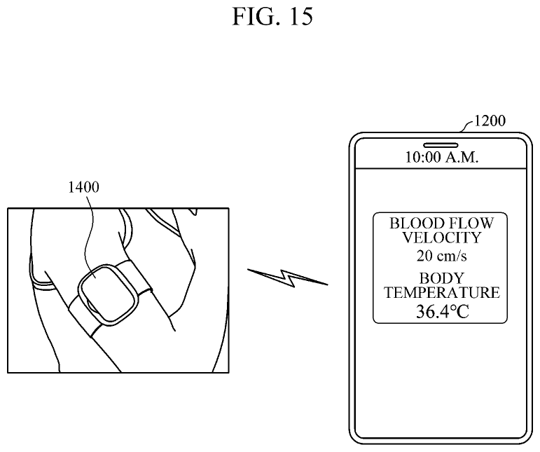

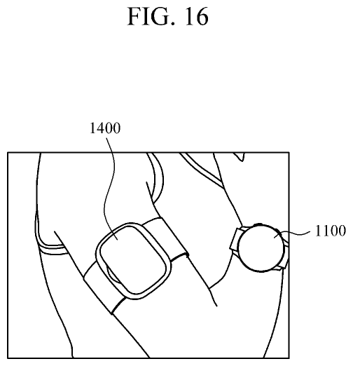

FIGS. 11 to 14 are diagrams illustrating examples of structures of an electronic device described above. FIGS. 15 and 16 are diagrams illustrating examples of obtaining blood flow information by using two or more devices operating in conjunction with each other.

The electronic device may include a sensor device, a processor, an input device, a communication module, a camera module, an output device, a storage device, and a power module. All the components of the electronic device may be integrally mounted in a specific device or may be distributed in two or more devices. The sensor device may include the heat source and the plurality of temperature sensors described above, and may further include an additional sensor, such as a gyro sensor, a Global Positioning System (GPS), and the like.

The processor may execute programs, stored in the storage device, to control components connected to the processor, and may perform various data processing or computation. For example, the processor may estimate blood flow information, including a blood temperature difference and blood flow velocity, based on temperature data measured on a surface of an object and temperature data measured at a position away from the object by using the plurality of temperature sensors of the sensor device. The processor may include a main processor, e.g., a central processing unit (CPU) or an application processor (AP), etc., and an auxiliary processor, e.g., a graphics processing unit (GPU), an image signal processor (ISP), a sensor hub processor, or a communication processor (CP), etc., which is operable independently from, or in conjunction with, the main processor.

The input device may receive a command and/or data to be used by each component of the electronic device, from a user and the like. The input device may include, for example, a microphone, a mouse, a keyboard, or a digital pen (e.g., a stylus pen, etc.).

The communication module may support establishment of a direct (e.g., wired) communication channel and/or a wireless communication channel between the electronic device and other electronic device, a server, or the sensor device within a network environment, and performing of communication via the established communication channel. The communication module may include one or more communication processors that are operable independently from the processor and supports a direct communication and/or a wireless communication. The communication module may include a wireless communication module, e.g., a cellular communication module, a short-range wireless communication module, or a global navigation satellite system (GNSS) communication module, etc., and/or a wired communication module, e.g., a local area network (LAN) communication module, a power line communication (PLC) module, and the like. These various types of communication modules may be integrated into a single chip, or may be separately implemented as multiple chips. The wireless communication module may identify and authenticate the electronic device in a communication network by using subscriber information (e.g., international mobile subscriber identity (IMSI), etc.) stored in a subscriber identification module.

The camera module may capture still images or moving images. The camera module may include a lens assembly having one or more lenses, image sensors, image signal processors, and/or flashes. The lens assembly included in the camera module may collect light emanating from a subject to be imaged.

The output device may visually/non-visually output data generated or processed by the electronic device. The output device may include a sound output device, a display device, an audio module, and/or a haptic module.

The sound output device may output sound signals to the outside of the electronic device. The sound output device may include a speaker and/or a receiver. The speaker may be used for general purposes, such as playing multimedia or playing record, and the receiver may be used for incoming calls. The receiver may be implemented separately from, or as part of, the speaker.

The display device may visually provide information to the outside of the electronic device. The display device may include, for example, a display, a hologram device, or a projector and control circuitry to control the devices. The display device may include touch circuitry adapted to detect a touch, and/or sensor circuitry (e.g., pressure sensor, etc.) adapted to measure the intensity of force incurred by the touch.

The audio module may convert a sound into an electrical signal or vice versa. The audio module may obtain the sound via the input device, or may output the sound via the sound output device, and/or a speaker and/or a headphone of another electronic device directly or wirelessly connected to the electronic device.

The haptic module may convert an electrical signal into a mechanical stimulus (e.g., vibration, motion, etc.) or electrical stimulus which may be recognized by a user by tactile sensation or kinesthetic sensation. The haptic module may include, for example, a motor, a piezoelectric element, and/or an electric stimulator.

The storage device may store operating conditions required for operating the sensor device, and various data necessary for other components of the electronic device. The various data may include, for example, input data and/or output data for software and instructions associated with the software, and the like. The storage device may include a volatile memory and/or a non-volatile memory.

The power module may control power supplied to the electronic device. The power module may be implemented as part of, for example, a power management integrated circuit (PMIC). The power module may include a battery, which may include a primary cell which is not rechargeable, a secondary cell which is rechargeable, and/or a fuel cell.

Referring to FIG. 11, the electronic device may be implemented as a wristwatch wearable device 1100, and may include a main body and a wrist strap. A display is provided on a front surface of the main body, and may display various application screens, including a blood temperature difference, blood flow information, warning information, time information, received message information, and the like. A sensor device 1110 may be disposed on a rear surface of the main body. A processor and various other components may be disposed in the main body. A heat source and a plurality of temperature sensors may be disposed in the sensor device 1110.

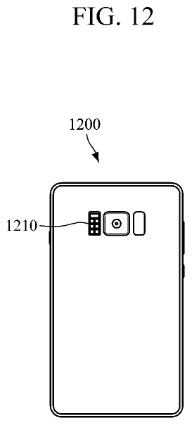

Referring to FIG. 12, the electronic device may be implemented as a mobile device 1200 such as a smartphone. The mobile device 1200 may include a housing and a display panel. The housing may form an exterior of the mobile device 1200. The housing has a first surface, on which a display panel and a cover glass may be disposed sequentially, and the display panel may be exposed to the outside through the cover glass. A sensor device 1210, a camera module and/or an infrared sensor, and the like may be disposed on a second surface of the housing. A processor and various other components may be disposed in the housing. A heat source and a plurality of temperature sensors may be disposed in the sensor device 1210.

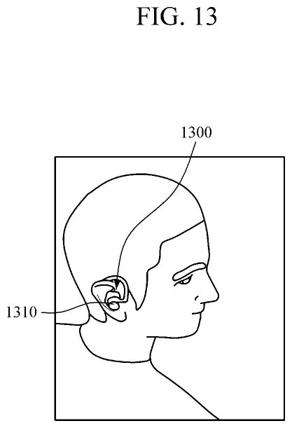

Referring to FIG. 13, the electronic device may be implemented as an ear-wearable device 1300. The ear-wearable device 1300 may include a main body and an ear strap. A user may wear the ear-wearable device 1300 by hanging the ear strap on the auricle. The ear strap may be omitted depending on a shape of the ear-wearable device 1300. The main body may be inserted into the external auditory meatus. A sensor device 1310 may be mounted in the main body at a contact portion with skin. The sensor device 1310 may include a heat source and a plurality of temperature sensors. Further, a processor, a communication device, and the like may be disposed in the main body.

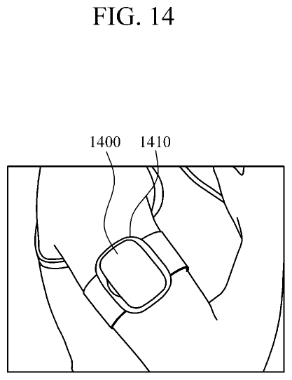

Referring to FIG. 14, an electronic device may be implemented as a patch-type wearable device 1400. The patch-type wearable device 1400 may have a contact pad to be attached to a surface (e.g., upper arm, wrist, etc.) of the object. In addition, a sensor board may be disposed therein, and a heat source and a plurality of temperature sensors may be mounted on the sensor board. Further, a processor, a communication device, and the like may be mounted in the patch-type wearable device 1400. The processor may obtain blood flow information based on temperature data measured by the plurality of temperature sensors.

FIG. 15 is a diagram illustrating an example of estimating blood flow information by using the patch-type wearable device 1400 and the mobile device 1200. As illustrated herein, when the patch-type wearable device 1400 is attached to the upper arm of the object and the sensor measures temperature in the vicinity of the upper arm, the patch-type wearable device 1400 may transmit the measured temperature data to the mobile device 1200. The mobile device 1200 may estimate blood flow information, e.g., blood flow velocity and/or core body temperature, by using the received temperature data, and may output an estimation result to a display. In this case, the patch-type wearable device 1400 may not include an algorithm for estimating the blood flow information.

FIG. 16 is a diagram illustrating an example of estimating blood flow information by using the patch-type wearable device 1400 and the smartwatch 1100. As illustrated herein, when the patch-type wearable device 1400 is attached to the upper arm of the object and the sensor measures temperature in the vicinity of the upper arm, the patch-type wearable device 1400 may transmit the measured temperature data to the smartwatch 1100. The smartwatch 1100 may estimate blood flow information in the vicinity of the upper arm by using the received temperature data. In this case, the patch-type wearable device 1400 may not include an algorithm for estimating the blood flow information. In addition, the smartwatch 1100 may measure temperature data from the wrist by using the sensor device 1110 mounted therein, and may estimate blood flow information in the vicinity of the wrist by using the measured temperature data. As described above, by simultaneously estimating the blood flow information in the vicinity of the upper arm and the wrist, estimation results at two positions, comparison data of the estimation results at two positions, or data obtained by integrating the estimation results at two positions, and the like may be provided to a user.

The present disclosure can be realized as a computer-readable code written on a computer-readable recording medium. The computer-readable recording medium may be any type of recording device in which data is stored in a computer-readable manner.

Examples of the computer-readable recording medium include a ROM, a RAM, a CD-ROM, a magnetic tape, a floppy disc, an optical data storage, and a carrier wave (e.g., data transmission through the Internet). The computer-readable recording medium can be distributed over a plurality of computer systems connected to a network so that a computer-readable code is written thereto and executed therefrom in a decentralized manner. Functional programs, codes, and code segments needed for realizing the present invention can be readily deduced by programmers of ordinary skill in the art to which the invention pertains.

The present disclosure has been described herein with regard to preferred embodiments. However, it will be obvious to those skilled in the art that various changes and modifications can be made without changing technical conception and essential features of the present disclosure. Thus, it is clear that the above-described embodiments are illustrative in all aspects and are not intended to limit the present disclosure.

What is claimed is:

1. An electronic device comprising:
a sensor comprising a heat source, a pair of first temperature sensors horizontally spaced apart from each other at two opposing sides of the heat source, and a pair of second temperature sensors vertically spaced apart from the pair of first temperature sensors in a thickness direction of the electronic device; and
at least one processor configured to:
control the heat source to generate heat while the electronic device is in contact with a user;
estimate a blood temperature difference between the two opposing sides of the heat source, based on temperatures measured by the pair of first temperature sensors and the pair of second temperature sensors; and
estimate blood flow information of the user based on the blood temperature difference.

2. The electronic device of claim 1, wherein the at least one processor is further configured to:
calculate a first temperature difference between the temperatures measured by the pair of first temperature sensors and a second temperature difference between the temperatures measured by the pair of second temperature sensors; and
estimate the blood temperature difference based on the first temperature difference and the second temperature difference.

3. The electronic device of claim 2, wherein the at least one processor is further configured to:
obtain an objective function that uses a relational expression between the first temperature difference and the blood temperature difference and a relational expression between the second temperature difference and the blood temperature difference; and
obtain the blood temperature difference at which the objective function is minimized.

4. The electronic device of claim 1, wherein the sensor further comprises a third temperature sensor horizontally spaced apart from the heat source,
wherein the at least one processor is further configured to correct the temperatures measured by the first temperature sensors based on temperature measured by the third temperature sensor.

5. The electronic device of claim 1, wherein the at least one processor is further configured to control an on-time period and an off-time period of the heat source according to a predetermined cycle,
wherein the first temperature sensors and the second temperature sensors are configured to measure temperatures during the off-time period of the heat source that follows the on-time period of the heat source.

6. The electronic device of claim 1, wherein the blood flow information comprises at least one of a blood flow velocity, a blood flow direction, a core body temperature, and a blood glucose change.

7. The electronic device of claim 6, wherein the at least one processor is further configured to estimate the blood flow velocity based on the blood temperature difference by using an estimation model that defines a correlation between the blood temperature difference and the blood flow velocity.

8. The electronic device of claim 6, wherein the at least one processor is further configured to:
estimate heat flux based on a difference between the temperatures measured by the first temperature sensor and the second temperature sensor which are disposed on at least one side of the heat source, and
estimate the core body temperature based on the estimated heat flux.

9. The electronic device of claim 6, wherein upon estimating the blood flow velocity, the at least one processor is further configured to:
calculate a difference between the estimated blood flow velocity and a reference blood flow velocity estimated at a reference time; and
predict a change in blood glucose compared to the reference time based on the difference between the estimated blood flow velocity and a reference blood flow velocity.

10. The electronic device of claim 1, wherein at least one of the first temperature sensors and the second temperature sensors is a thermistor.

11. The electronic device of claim 1, wherein the heat source has a diameter of 1 mm to 10 mm.

12. The electronic device of claim 1, wherein a distance between a center and the two opposing sides of the heat source is within 8.4 mm.

13. The electronic device of claim 1, wherein a distance between the pair of first temperature sensors and the pair of second temperature sensors is 0.4 mm to 10 mm.

14. The electronic device of claim 1, wherein the sensor further comprises a thermally insulating materials disposed between the first temperature sensors and between the second temperature sensors.

15. A method of estimating blood flow information by an electronic device, the method comprising:
controlling a heat source to generate heat while the electronic device is in contact with a user;
measuring temperatures by using a pair of first temperature sensors horizontally spaced apart from each other at two opposing sides of the heat source, and a pair of second temperature sensors vertically spaced apart from the pair of first temperature sensors in a thickness direction of the electronic device;
estimating a blood temperature difference between the two opposing sides of the heat source, based on the temperatures measured by the pair of first temperature sensors and the pair of second temperature sensors; and
estimating blood flow information of the user based on the blood temperature difference.

16. The method of claim 15, wherein the estimating of the blood temperature difference comprises:
calculating a first temperature difference between the temperatures measured by the pair of first temperature sensors and a second temperature difference between the temperatures measured by the pair of second temperature sensors; and
estimating the blood temperature difference based on the first temperature difference and the second temperature difference.

17. The method of claim 15, further comprising measuring a temperature by using a third temperature sensor horizontally spaced apart from the heat source,
wherein the estimating of the blood temperature difference comprises correcting the temperatures measured by the first temperature sensors based on the temperature measured by the third temperature sensor.

18. The method of claim 15, wherein the controlling of the heat source comprises:
controlling an on-time period and an off-time period of the heat source according to a predetermined cycle,
wherein the measuring of the temperatures comprises measuring temperatures during the off-time period of the heat source that follows the on-time period of the heat source.

19. The method of claim 15, wherein the estimating of the blood flow information comprises estimating a blood flow velocity, included in the blood flow information, based on the estimated blood temperature difference by using an estimation model that defines a correlation between the blood temperature difference and the blood flow velocity.

20. A patch-type sensor comprising:
a contact pad configured to make contact with a skin surface;
a heat source configured to apply heat to the skin surface when the contact pad makes contact with the skin surface;
a pair of first temperature sensors horizontally spaced apart from each other at two opposing sides of the heat source;
a pair of second temperature sensors vertically spaced apart from the pair of first temperature sensors in a depth direction of the patch-type sensor, and
a communication interface configured to transmit temperature data, measured by the pair of first temperature sensors and the pair of the second temperature sensors, to an external electronic device, to enable the external electronic device to obtain blood flow information by estimating a blood temperature difference based on the temperature data.
